(19) United States
(12) Patent Application Publication
(10) Pub. No.: US 2009/0053318 A1
(43) Pub. Date: Feb. 26, 2009

(54) Forming Embolic Particles

(76) Inventors: Sharon Mi Lyn Tan, Boston, MA (US); Sonali Puri, Ashland, MA (US); Robert E. Richard, Wrentham, MA (US)

Correspondence Address: FISH & RICHARDSON PC, P.O. BOX 1022, MINNEAPOLIS, MN, 55440-1022, US

(21) Appl. No.: 12/194,855

(22) Filed: Aug. 20, 2008

Related U.S. Application Data
(60) Provisional application No. 60/957,047, filed on Aug. 21, 2007.

(51) Int. Cl. A61K 9/14; C08F 8/12; C08F 6/00

(52) U.S. Cl. 424/501; 525/62; 522/153

(57) ABSTRACT

Methods of making polymer particles, as well as related particles, compositions, and methods are disclosed.

### CROSS-REFERENCE TO RELATED APPLICATION

This application is a utility from provisional of and claims priority under 35 U.S.C. §120 to U.S. Application Ser. No. 60/957,047, filed Aug. 21, 2007, the entire contents of which being hereby fully incorporated by reference.

### TECHNICAL FIELD

This disclosure relates to methods of making polymer particles, as well as related particles, compositions, and methods.

### BACKGROUND

Agents, such as therapeutic agents, can be delivered systemically, for example, by injection through the vascular system or oral ingestion, or they can be applied directly to a site where treatment is desired. In some cases, particles are used to deliver a therapeutic agent to a target site. Additionally or alternatively, particles may be used to perform embolization procedures and/or to perform radiotherapy procedures.

### SUMMARY

In one aspect, a method comprises freeze-drying a mixture comprising a polymer to form a particle. Embodiments can include one or more of the following features.

In some embodiments, the polymer includes polyvinyl alcohol.

In some embodiments, freeze-drying the mixture includes reducing a pressure applied to the mixture to less than 500 millitorrs (mT). In some cases, reducing the pressure applied to the mixture includes reducing the pressure applied to the mixture to less than 500 mT and more than 10 mT.

In some embodiments, freeze-drying the mixture includes reducing the temperature applied to the mixture to less than −20° C. In some cases, freeze-drying the mixture includes reducing the temperature applied to the mixture to less than −50° C. and more than −85° C.

In some embodiments, methods also include freeze-drying the mixture at least two times (e.g., at least three times, at least four times, at least five times).

In some embodiments, freeze-drying the mixture includes iteratively freeze-drying and thawing the mixture until the moisture content of the mixture is less than 5 percent (e.g., less than 1 percent).

In some embodiments, methods also include adding a therapeutic agent to the particle. In some cases, adding the therapeutic agent to the particle includes disposing the therapeutic agent into pores of the particle after the particle is formed. In some cases, adding the therapeutic agent to the particle includes adding the therapeutic agent to the mixture before freezing the mixture. In some cases, adding the therapeutic agent to the particle includes coating the particle with the therapeutic agent after the particle is formed.

In some embodiments, methods also include adding a second polymer to the mixture.

In some embodiments, methods also include irradiating the mixture.

In another aspect, a method includes forming a particle having a maximum dimension of 5,000 microns by irradiating a polymer to crosslink the polymer. Embodiments can include one or more of the following features.

In some embodiments, methods also include freeze-drying the polymer.

In some embodiments, irradiating the polymer includes irradiating the polymer with e-beam radiation.

In some embodiments, irradiating the polymer includes irradiating the polymer with gamma radiation.

In some embodiments, irradiating the polymer includes applying a dose of radiation from 20 to 110 kilograys (e.g., from 25 to 100 kilograys) to the polymer.

In some embodiments, methods also include forming microspheres comprising the polymer before irradiating the polymer.

In some embodiments, methods also include adding a therapeutic agent to the particle. In some cases, adding the therapeutic agent to the particle includes disposing the therapeutic agent into pores of the particle after the particle is formed. In some cases, adding the therapeutic agent to the particle includes mixing the therapeutic agent with the polymer before irradiating the polymer. In some cases, adding the therapeutic agent to the particle includes coating the particle with the therapeutic agent after the particle is formed.

In some embodiments, methods also include placing the polymer in a mold to form a polymer sheet before irradiating the polymer. In some cases, methods also include grinding the polymer sheet to form particles after irradiating the polymer sheet.

In some embodiments, methods also include placing the polymer in a container and purging oxygen from the container before irradiating the polymer.

Embodiments can include one or more of the following advantages.

Freeze-drying and/or irradiation techniques can be used to form polymer particles without using chemical agents and acids (e.g., glutaraldehyde, formaldehyde, and sulfuric acid) to induce cross-linking in the polymer. Such chemical agents typically need to be removed from the polymer particles before use in biomedical applications. Moreover, therapeutic agents can be added to the particles at the time of particle formation rather than after cross-linking.

The particles can optionally be used to deliver therapeutic agents within a body lumen, alone or in combination with an embolization procedure.

The at least partially crystalline polymer can render the particle(s) relatively stable (e.g., insoluble) in vivo.

The methods can provide particles appropriate for use in, for example, embolization and/or therapeutic agent delivery within a body lumen (e.g., a blood vessel of a human or an animal).

The methods can be relatively gentle so that an additive, such as therapeutic agents, can be provided in the particle before and/or during the crystallizing of the polymer with little or no undesirable chemical reaction involving the additive occurring during the crystallizing process.

The methods can provide particles having certain desirable physical properties for delivery in a body lumen (e.g., a blood vessel), such as, for example, hardness.

Features and advantages are in the description, drawings, and claims.

### DESCRIPTION OF DRAWINGS

FIGS. 1A and 1B are, respectively, a side view and a cross-section of an embodiment of a particle.

FIGS. 2A and 2B are an illustration of an embodiment of a system and method for producing particles.

FIG. 3 is an illustration of a droplet generator system.

FIG. 4 is an illustration of an embodiment of a system and method for producing particles.

FIG. 5 is an illustration of an embodiment of a system and method for producing particles.

FIG. 6A is a schematic illustrating an embodiment of a method of injecting a composition including particles into a vessel.

FIG. 6B is a greatly enlarged view of region 6B in FIG. 6A.

FIG. 7 is an embodiment of a particle.

Like reference symbols in the various drawings indicate like elements.

### DETAILED DESCRIPTION

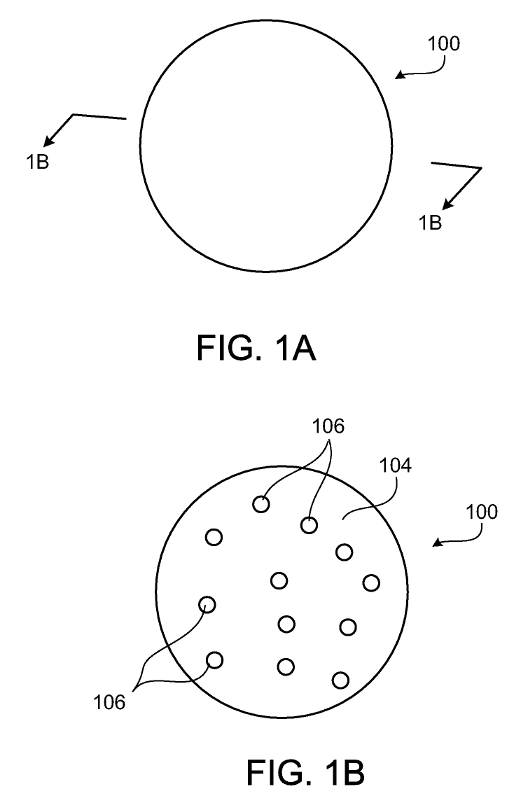

FIGS. 1A and 1B show a particle 100 that can be used, for example, in an embolization procedure. Particle 100 includes a polymer matrix 104 including pores 106. The matrix 104 is formed of a polymer, such as polyvinyl alcohol (PVA).

Generally, the polymer from which matrix 104 is formed is at least partially crystalline. For example, the polymer can be at least 2% (e.g., at least 3%, at least 4%, at least 5%, at least 10%) crystalline. As used herein, the degree that a polymer is crystalline is measured using differential scanning calorimetry, X-ray diffraction or density measurements.

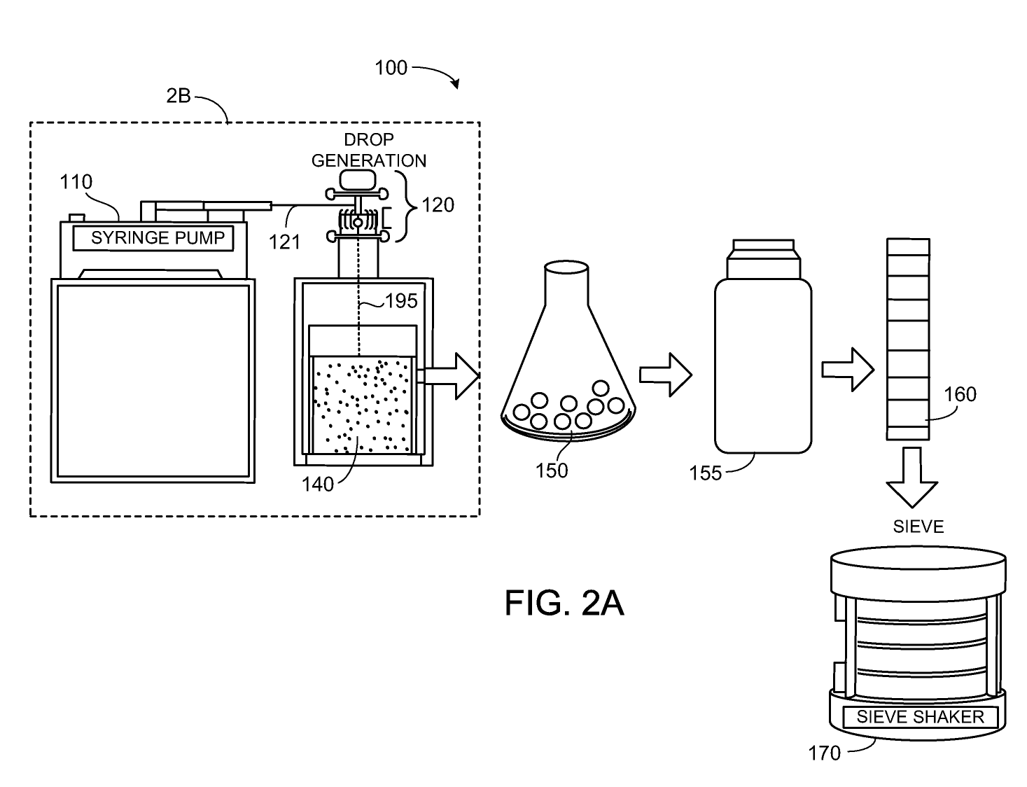

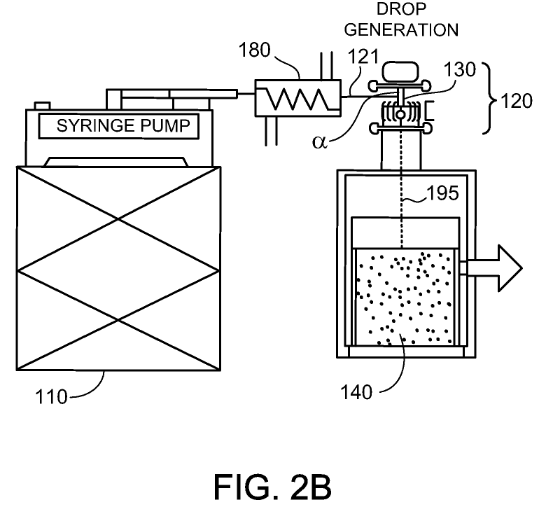

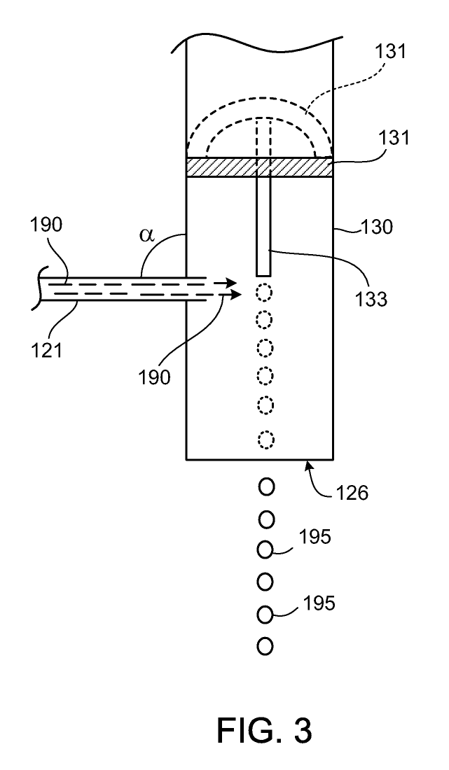

FIGS. 2A, 2B, and 3 show a system 100 for producing particles. System 100 includes a flow controller 110, a drop generator 120 including a nozzle 130, a gelling vessel 140, a cooling vessel 150, a freeze-dryer 155, an optional gel dissolution chamber 160, and a filter 170. An example of a commercially available drop generator is the model NISCO Encapsulation unit VAR D (NISCO Engineering, Zurich, Switzerland). An example of commercially available freeze-dryer is the Opti-Dry Bench-top Freeze-Dryer, Millrock Technology, Kingston, N.Y.

Flow controller 110 includes a high pressure pumping apparatus, such as a syringe pump (e.g., model PHD4400, Harvard Apparatus, Holliston, Mass.). Flow controller 110 delivers a stream 190 of a solution including a polymer and a gelling precursor to a viscosity controller 180, which heats the solution to reduce its viscosity prior to delivery to drop generator 120. In some embodiments, a therapeutic agent can be added before the solution is delivered to viscosity controller 180. Viscosity controller 180 is connected to nozzle 130 of drop generator 120 via tubing 121. After stream 190 has traveled from flow controller 180 through tubing 121, stream 190 flows around a corner having an angle a, and enters nozzle 130. As shown, angle α is about 90 degrees. However, in some embodiments, angle a can be less than 90 degrees (e.g., less than about 70 degrees, less than about 50 degrees, less than about 30 degrees).

As stream 190 enters nozzle 130, a membrane 131 in nozzle 130 is subjected to a periodic disturbance (a vibration). The vibration causes membrane 131 to pulse upward (to the position shown in phantom in FIG. 3) and then return back to its original position. Membrane 131 is connected to a rod 133 that transmits the vibration of membrane 131, thereby periodically disrupting the flow of stream 190 as stream 190 enters nozzle 130. This periodic disruption of stream 190 causes stream 190 to form drops 195. Drops 195 fall into gelling vessel 140, where drops 195 are stabilized by gel formation. During gel formation, the gelling precursor in drops 195 is converted from a solution to a gel form by a gelling agent contained in gelling vessel 140. The gel-stabilized drops are then transferred from gelling vessel 140 to cooling vessel 150 (e.g., a freeze-drying flask).

In a freeze-drying cycle, the gel-stabilized drops are cooled to a temperature below the eutectic point of the polymer. The eutectic point of the polymer is the lowest temperature at which the solid and liquid phase of the polymer can coexist. The frozen drops are then transferred to freeze-dryer 155. In freeze-dryer 155, the pressure is lowered and enough heat is applied the drops to sublimate water contained in the drops. Microscopic pores can be formed in the drops as water molecules change from solid phase directly to gas phase and exit the drops. Freeze-drying can also induce at least partial crystallization of the polymer to form particles. The particles are subsequently thawed. The freeze-drying cycle can be repeated as desired to obtain, for example, a desired degree of crystallinity of the polymer. In some embodiments, the freeze-drying cycle can be performed at least two times (e.g., at least three times, at least four times, at least five times). In some embodiments, the particles are frozen before being placed in a freeze-dryer.

In some embodiments, a polymer such as, for example, PVA, is mixed with another polymer or polymers that exhibit a lower degree of crystallinity in response to freeze-drying before drop generation.

In some embodiments, when in cooling vessel 150, the particles are subjected to a temperature reduced to less than 15° C. (e.g., less than 10° C., less than 0° C., less than −15° C., less than −25° C., less than −35° C., less than −50° C., less than −60° C., less than −80° C.). For example, when in cooling vessel 150, the particles are reduced to a temperature of from −100° C. to −50° C. (e.g., from −90° C. to −60° C., from −90° C. to −80° C.). In certain embodiments, when in cooling vessel 150, the particles are at a temperature of −85° C.

In certain embodiments, the particles are held in cooling vessel 150 at reduced temperature for at least 10 minutes (e.g., at least 30 minutes, at least one hour, at least two hours, at least five hours, at least 10 hours, at least 20 hours, at least one day) and/or at most one week (e.g., at most three days, at most two days, at most one day). For example, the particles can be held in cooling vessel 150 at reduced temperature for from one hour to two days (e.g., from five hours to two days, from 10 hours to two days). In some embodiments, the particles are held in cooling vessel 150 at reduced temperature for one day.

In some embodiments, cooling vessel 150 is a refrigerator or a freezer. In some embodiments, cooling vessel 150 is a container holding a coolant (e.g., liquid nitrogen, liquid carbon dioxide). The particles can be immersed directly in the coolant or can be placed in a secondary container (e.g., a glass vial) which is immersed in the coolant for a period of time (e.g., one minute to one hour, two minutes to 30 minutes, three minutes to 10 minutes, five minutes).

In some embodiments, freeze-dryer 155 can be used to reduce the moisture content of the particles to less than 10 percent (e.g., less than 5 percent, less than 1 percent). Freeze-dryer 155 can be operated at temperatures below 0° C. (e.g., below −20° C., below −25° C., below −50° C., below −80° C., below −100° C., below −150° C., below −200° C.) and/or at temperatures above −210° C. (e.g., above −175° C., above −125° C., above −100° C., above −50° C.). For example, freeze-dryer 155 can be operated at temperatures from −50° C. to −100° C. (e.g., about −85° C.). Freeze-dryer 155 can be operated at vacuum pressures from 10 mT to 500 mT. For example, freeze-dryer 155 can be operated at vacuum pressures from 200 mT mm Hg to 500 mT.

After the freeze-drying cycle(s), the particles can optionally be transferred to gel dissolution chamber 160. In gel dissolution chamber 160, the gelling precursor (which was converted to a gel) in the particles is dissolved. After the particle formation process has been completed, the particles can be filtered in filter 170 to remove debris, and sterilized and packaged as a composition including particles.

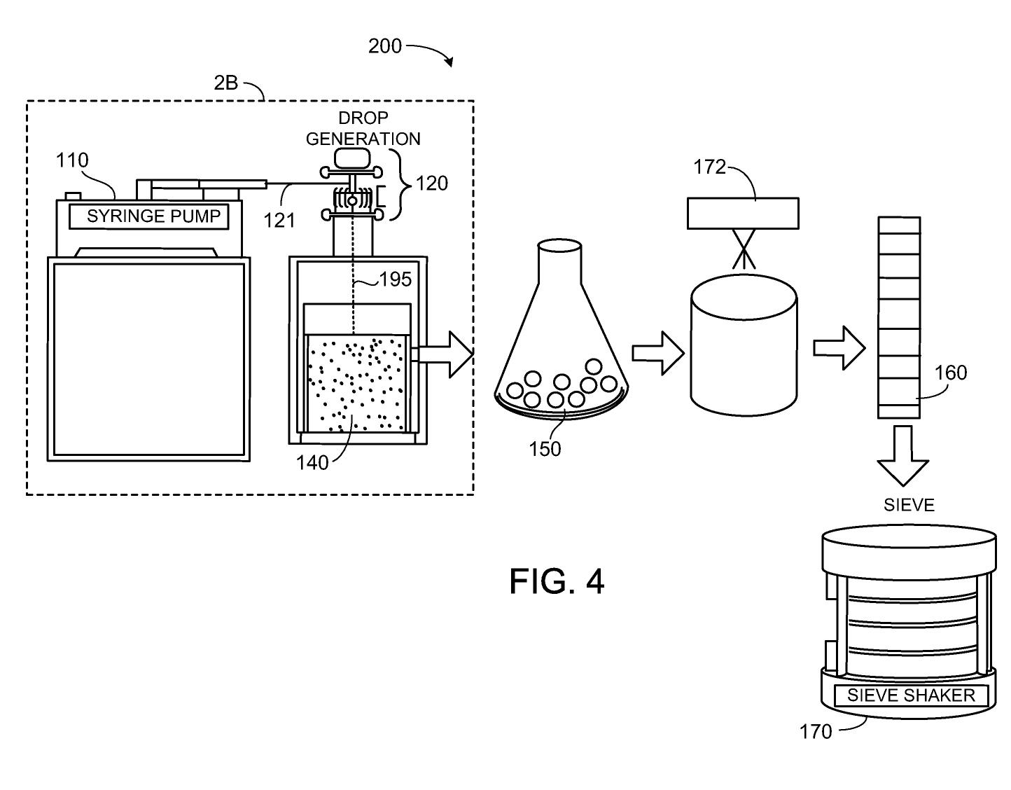

FIG. 4 shows a system 200 for producing particles. Like system 100, system 200 includes flow controller 110, drop generator 120 including nozzle 130, gelling vessel 140, cooling vessel 150, optional gel dissolution chamber 160, and filter 170. However, system 200 includes a radiation source 172 rather freeze-dryer 155. An example of commercially available radiation source is the 5 MeV E-beam Sterilizer system, Mevex, Ontario, Canada.

System 200 generates gel-stabilized drops using the same equipment and approach as system 100. The gel-stabilized drops are placed in a glass vial which is immersed in liquid nitrogen to freeze the drops. After freezing, nitrogen purging is optionally used to remove oxygen from the glass vial containing the frozen drops. Without wishing to be bound by theory, it is believed that oxygen can scavenge radicals involved cross-link formation and reduce the degree of cross-linking induced by irradiating the polymer. The frozen drops are then transferred to radiation source 172 and irradiated with a dose of radiation to form particles.

After irradiation, the particles can optionally be transferred to gel dissolution chamber 160. In gel dissolution chamber 160, the gelling precursor (which was converted to a gel) in the particles is dissolved. After the particle formation process has been completed, the particles can be filtered in filter 170 to remove debris, and sterilized and packaged as a composition including particles.

In some embodiments, radiation source 172 can emit, for example, electron beam radiation or gamma radiation. Radiation source 172 is operated to provide a radiation dose of more than 10 kilograys, (e.g., more than 20 kilograys, more than 40 kilograys, more than 60 kilograys, more than 80 kilograys, more than 100 kilograys) and/or less than 150 kilograys (e.g., less than 110 kilograys, less than 90 kilograys, less than 70 kilograys, less than 50 kilograys, less than 30 kilograys). For example, in some embodiments, radiation source 172 is operated to provide a radiation dose of from 20 to 110 kilograys (e.g., 25 kilograys, 50 kilograys, 75 kilograys, 100 kilograys).

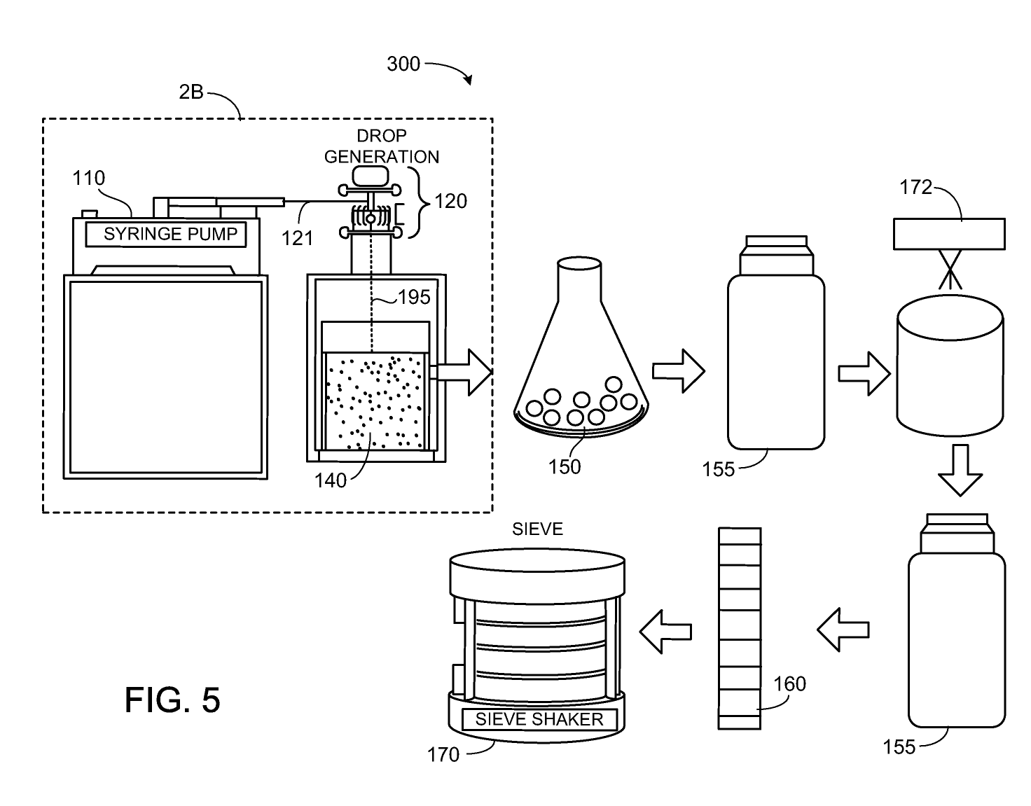

FIG. 5 shows a system 300 for producing particles. Like system 100 and system 200, system 300 includes flow controller 110, drop generator 120 including nozzle 130, gelling vessel 140, cooling vessel 150, optional gel dissolution chamber 160, and filter 170. However, system 300 includes both freeze-dryer 155 and radiation source 172.

System 300 generates gel-stabilized drops using the same equipment and approach as system 100 and system 200. The gel-stabilized drops placed in a glass vial which is immersed in liquid nitrogen to freeze the drops. After freezing, nitrogen purging is optionally used to remove oxygen from the glass vial containing the frozen drops. The frozen drops are then transferred to freeze-dryer 155 for at least one freeze-drying cycle. After freeze-drying, the drops are transferred to radiation source 172 and irradiated with a dose of radiation (e.g., electron beam radiation, gamma radiation) to form particles. The irradiated particles are then transferred back to freeze-dryer 155 for at least one additional freeze-drying cycle. Optionally, the particles can then be transferred to gel dissolution chamber 160. In gel dissolution chamber 160, the gelling precursor (which was converted to a gel) in the particles is dissolved. After the particle formation process has been completed, the particles can be filtered in filter 170 to remove debris, and sterilized and packaged as a composition including particles.

Drop generators are described, for example, in Lanphere et al., U.S. Patent Application Publication No. US 2004/0096662 A1, published on May 20, 2004, and entitled “Embolization”, and in DiCarlo et al., U.S. patent application Ser. No. 11/111,511, filed on Apr. 21, 2005, and entitled “Particles”, both of which are incorporated herein by reference.

In general, the maximum dimension of particle 100 is 5,000 microns or less (e.g., from two microns to 5,000 microns; from 10 microns to 5,000 microns; from 40 microns to 2,000 microns; from 100 microns to 700 microns; from 500 microns to 700 microns; from 100 microns to 500 microns; from 100 microns to 300 microns; from 300 microns to 500 microns; from 500 microns to 1,200 microns; from 500 microns to 700 microns; from 700 microns to 900 microns; from 900 microns to 1,200 microns; from 1,000 microns to 1,200 microns). In some embodiments, the maximum dimension of particle 100 is 5,000 microns or less (e.g., 4,500 microns or less, 4,000 microns or less, 3,500 microns or less, 3,000 microns or less, 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, the maximum dimension of particle 100 is less than 100 microns (e.g., less than 50 microns).

In some embodiments, particle 100 can be substantially spherical. In certain embodiments, particle 100 can have a sphericity of 0.8 or more (e.g., 0.85 or more, 0.9 or more, 0.95 or more, 0.97 or more). Particle 100 can be, for example, manually compressed, essentially flattened, while wet to 50 percent or less of its original diameter and then, upon exposure to fluid, regain a sphericity of 0.8 or more (e.g., 0.85 or more, 0.9 or more, 0.95 or more, 0.97 or more). The sphericity of a particle can be determined using a Beckman Coulter RapidVUE Image Analyzer version 2.06 (Beckman Coulter, Miami, Fla.). Briefly, the RapidVUE takes an image of continuous-tone (gray-scale) form and converts it to a digital form through the process of sampling and quantization. The system software identifies and measures particles in an image in the form of a fiber, rod or sphere. The sphericity of a particle, which is computed as Da/Dp (where Da=√(4A/π); Dp=P/π; A=pixel area; P=pixel perimeter), is a value from zero to one, with one representing a perfect circle.

Examples of polymers include polymers that include vinyl alcohol monomers, vinyl formal monomers and/or vinyl acetate monomers. As referred to herein, a vinyl formal monomer unit has the following structure:

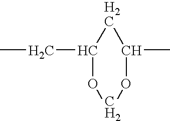

As referred to herein, a vinyl alcohol monomer unit has the following structure:

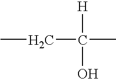

As referred to herein, a vinyl acetate monomer unit has the following structure:

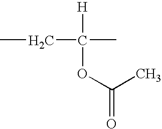

In general, the monomer units can be arranged in a variety of different ways. As an example, in some embodiments, the polymer can include different monomer units that alternate with each other. For example, the polymer can include repeating blocks, each block including a vinyl formal monomer unit, a vinyl alcohol monomer unit, and a vinyl acetate monomer unit. As another example, in certain embodiments, the polymer can include blocks including multiple monomer units of the same type. Generally, however, there should be sufficient PVA present in the polymer to allow the polymer to crystallize.

In some embodiments, the polymer can have the formula that is schematically represented below, in which x, y and z each are integers that are greater than zero. In certain embodiments, x is zero. The individual monomer units that are shown can be directly attached to each other, and/or can include one or more other monomer units (e.g., vinyl formal monomer units, vinyl alcohol monomer units, vinyl acetate monomer units) between them:

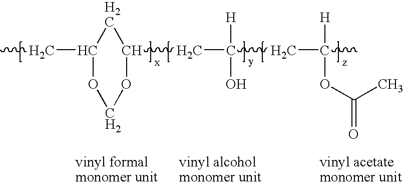

Optionally, formal linkages can occur between PVA molecules giving crosslinks.

In some embodiments, the polymer can include at least five percent by weight (e.g., at least 15 percent by weight, at least 25 percent by weight, at least 35 percent by weight) vinyl alcohol monomer units, and/or at most 80 percent by weight (e.g., at most 50 percent by weight, at most 25 percent by weight, at most 10 percent by weight) vinyl alcohol monomer units. The weight percent of a monomer unit in a polymer can be measured using solid-state NMR spectroscopy.

Generally, the polymer will contain little or no vinyl formal monomer units. In some embodiments, the polymer can include at most 10 percent by weight (e.g., at most 5 percent by weight, at most 2 percent by percent by weight) vinyl formal monomer units and/or at least 0.1 percent by weight (e.g., at least 0.5 percent by weight, at least 1 percent by weight) vinyl formal monomer units. As used herein, the weight percent of a monomer unit in a polymer is measured using solid-state NMR spectroscopy as described above.

In some embodiments, the polymer can include at least one percent by weight (e.g., at least two percent by weight, at least five percent by weight, at least 10 percent by weight, at least 15 percent by weight) vinyl acetate monomer units, and/or at most 20 percent by weight (e.g., at most 15 percent by weight, at most 10 percent by weight, at most five percent by weight) vinyl acetate monomer units. As used herein, the weight percent of a monomer unit in a polymer is measured using solid-state NMR spectroscopy as described above.

Other polymers may also be used as a matrix polymer in particle 10. Examples of polymers include polyacrylic acids, polymethacrylic acids, poly vinyl sulfonates, carboxymethyl celluloses, hydroxyethyl celluloses, substituted celluloses, polyacrylamides, polyethylene glycols, polyamides, polyureas, polyurethanes, polyesters, polyethers, polystyrenes, polysaccharides, polylactic acids, polyethylenes, polymethylmethacrylates, polycaprolactones, polyglycolic acids, poly(lactic-co-glycolic) acids (e.g., poly(d-lactic-co-glycolic) acids) and copolymers or mixtures thereof Polymers are described, for example, in Lanphere et al., U.S. Patent Application Publication No. US 2004/0096662 A1, published on May 20, 2004, and entitled “Embolization“; Song et al., U.S. patent application Ser. No. 11/314,056, filed on Dec. 21, 2005, and entitled “Block Copolymer Particles”; and Song et al., U.S. patent application Ser. No. 11/314,557, filed on Dec. 21, 2005, and entitled “Block Copolymer Particles”, all of which are incorporated herein by reference.

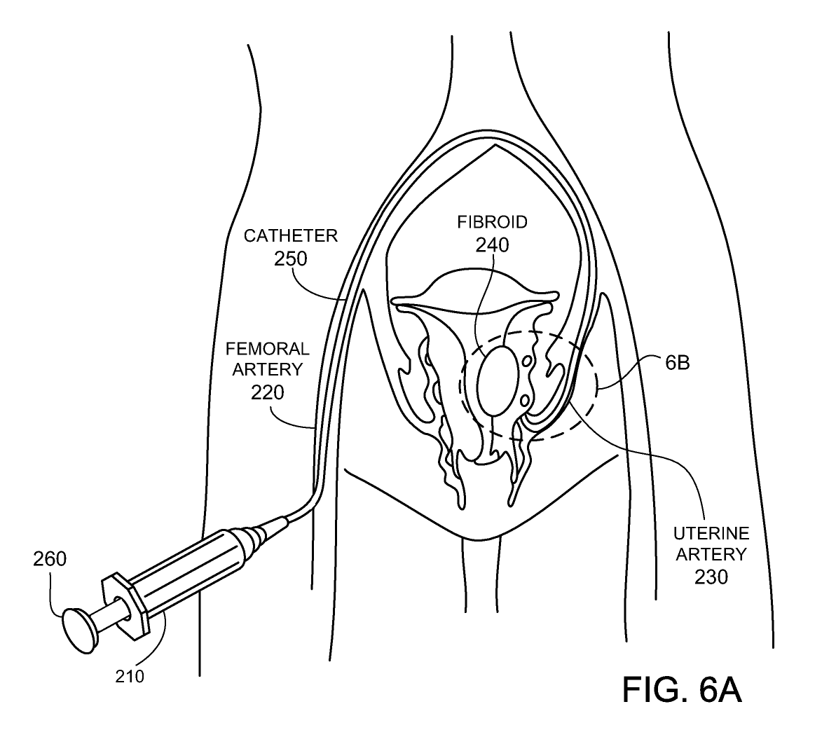

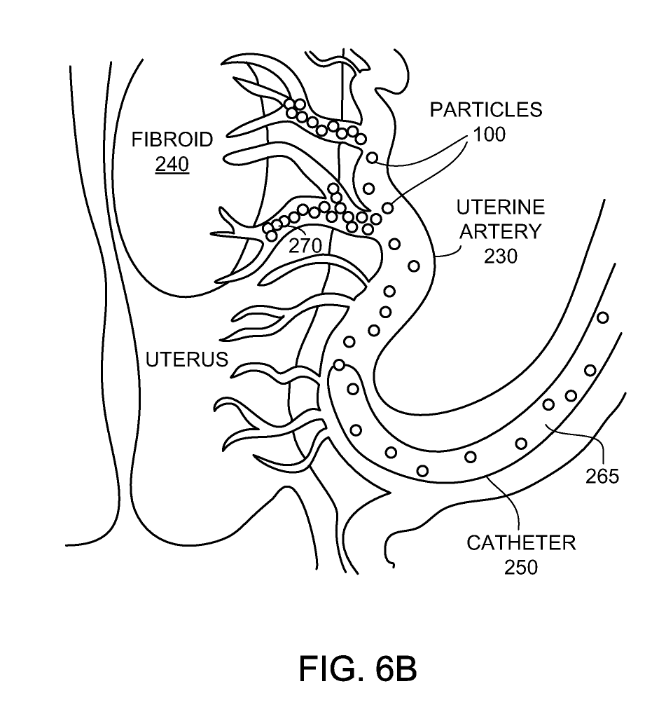

Multiple particles can be combined with a carrier fluid (e.g., a pharmaceutically acceptable carrier, such as a saline solution, a contrast agent, or both) to form a composition, which can then be delivered to a site and used to embolize the site. FIGS. 6A and 6B illustrate the use of a composition including particles to embolize a lumen of a subject. As shown, a composition including particles 100 and a carrier fluid is injected into a vessel through an instrument such as a catheter 250. Catheter 250 is connected to a syringe barrel 210 with a plunger 260. Catheter 250 is inserted, for example, into a femoral artery 220 of a subject. Catheter 250 delivers the composition to, for example, occlude a uterine artery 230 leading to a fibroid 240 located in the uterus of a female subject. The composition is initially loaded into syringe 210. Plunger 260 of syringe 210 is then compressed to deliver the composition through catheter 250 into a lumen 265 of uterine artery 230.

FIG. 6B, which is an enlarged view of section 6B of FIG. 6A, shows uterine artery 230, which is subdivided into smaller uterine vessels 270 (e.g., having a diameter of two millimeters or less) that feed fibroid 240. The particles 100 in the composition partially or totally fill the lumen of uterine artery 230, either partially or completely occluding the lumen of the uterine artery 230 that feeds uterine fibroid 240.

Compositions including particles such as particles 100 can be delivered to various sites in the body, including, for example, sites having cancerous lesions, such as the breast, prostate, lung, thyroid, or ovaries. The compositions can be used in, for example, neural, pulmonary, and/or AAA (abdominal aortic aneurysm) applications. The compositions can be used in the treatment of, for example, fibroids, tumors, internal bleeding, arteriovenous malformations (AVMs), and/or hypervascular tumors. The compositions can be used as, for example, fillers for aneurysm sacs, AAA sac (Type II endoleaks), endoleak sealants, arterial sealants, and/or puncture sealants, and/or can be used to provide occlusion of other lumens such as fallopian tubes. Fibroids can include uterine fibroids which grow within the uterine wall (intramural type), on the outside of the uterus (subserosal type), inside the uterine cavity (submucosal type), between the layers of broad ligament supporting the uterus (interligamentous type), attached to another organ (parasitic type), or on a mushroom-like stalk (pedunculated type). Internal bleeding includes gastrointestinal, urinary, renal and varicose bleeding. AVMs are, for example, abnormal collections of blood vessels (e.g. in the brain) which shunt blood from a high pressure artery to a low pressure vein, resulting in hypoxia and malnutrition of those regions from which the blood is diverted. In some embodiments, a composition containing the particles can be used to prophylactically treat a condition.

The magnitude of a dose of a composition can vary based on the nature, location and severity of the condition to be treated, as well as the route of administration. A physician treating the condition, disease or disorder can determine an effective amount of composition. An effective amount of embolic composition refers to the amount sufficient to result in amelioration of symptoms and/or a prolongation of survival of the subject, or the amount sufficient to prophylactically treat a subject. The compositions can be administered as pharmaceutically acceptable compositions to a subject in any therapeutically acceptable dosage, including those administered to a subject intravenously, subcutaneously, percutaneously, intratrachealy, intramuscularly, intramucosaly, intracutaneously, intra-articularly, orally or parenterally.

A composition can include a mixture of particles (e.g., particles formed of polymers including different weight percents of vinyl alcohol monomer units, particles including different types of therapeutic agents), or can include particles that are all of the same type. In some embodiments, a composition can be prepared with a calibrated concentration of particles for ease of delivery by a physician. A physician can select a composition of a particular concentration based on, for example, the type of procedure to be performed. In certain embodiments, a physician can use a composition with a relatively high concentration of particles during one part of an embolization procedure, and a composition with a relatively low concentration of particles during another part of the embolization procedure.

Suspensions of particles in saline solution can be prepared to remain stable (e.g., to remain suspended in solution and not settle and/or float) over a desired period of time. A suspension of particles can be stable, for example, for from one minute to 20 minutes (e.g. from one minute to 10 minutes, from two minutes to seven minutes, from three minutes to six minutes).

In some embodiments, particles can be suspended in a physiological solution by matching the density of the solution to the density of the particles. In certain embodiments, the particles and/or the physiological solution can have a density of from one gram per cubic centimeter to 1.5 grams per cubic centimeter (e.g., from 1.2 grams per cubic centimeter to 1.4 grams per cubic centimeter, from 1.2 grams per cubic centimeter to 1.3 grams per cubic centimeter).

In certain embodiments, the carrier fluid of a composition can include a surfactant. The surfactant can help the particles to mix evenly in the carrier fluid and/or can decrease the likelihood of the occlusion of a delivery device (e.g., a catheter) by the particles. In certain embodiments, the surfactant can enhance delivery of the composition (e.g., by enhancing the wetting properties of the particles and facilitating the passage of the particles through a delivery device). In some embodiments, the surfactant can decrease the occurrence of air entrapment by the particles in a composition (e.g., by porous particles in a composition). Examples of liquid surfactants include Tween® 80 (available from Sigma-Aldrich) and Cremophor EL® (available from Sigma-Aldrich). An example of a powder surfactant is Pluronic® F127 NF (available from BASF). In certain embodiments, a composition can include from 0.05 percent by weight to one percent by weight (e.g., 0.1 percent by weight, 0.5 percent by weight) of a surfactant. A surfactant can be added to the carrier fluid prior to mixing with the particles and/or can be added to the particles prior to mixing with the carrier fluid.

In some embodiments, among the particles delivered to a subject (e.g., in a composition), the majority (e.g., 50 percent or more, 60 percent or more, 70 percent or more, 80 percent or more, 90 percent or more) of the particles can have a maximum dimension of 5,000 microns or less (e.g., 4,500 microns or less, 4,000 microns or less, 3,500 microns or less, 3,000 microns or less, 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, among the particles delivered to a subject, the majority of the particles can have a maximum dimension of less than 100 microns (e.g., less than 50 microns).

In certain embodiments, the particles delivered to a subject (e.g., in a composition) can have an arithmetic mean maximum dimension of 5,000 microns or less (e.g., 4,500 microns or less, 4,000 microns or less, 3,500 microns or less, 3,000 microns or less, 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, the particles delivered to a subject can have an arithmetic mean maximum dimension of less than 100 microns (e.g., less than 50 microns).

Exemplary ranges for the arithmetic mean maximum dimension of particles delivered to a subject include from 100 microns to 500 microns; from 100 microns to 300 microns; from 300 microns to 500 microns; from 500 microns to 700 microns; from 700 microns to 900 microns; from 900 microns to 1,200 microns; and from 1,000 microns to 1,200 microns. In general, the particles delivered to a subject (e.g., in a composition) can have an arithmetic mean maximum dimension in approximately the middle of the range of the diameters of the individual particles, and a variance of 20 percent or less (e.g. 15 percent or less, 10 percent or less).

In some embodiments, the arithmetic mean maximum dimension of the particles delivered to a subject (e.g., in a composition) can vary depending upon the particular condition to be treated. As an example, in certain embodiments in which the particles are used to embolize a liver tumor, the particles delivered to the subject can have an arithmetic mean maximum dimension of 500 microns or less (e.g., from 100 microns to 300 microns; from 300 microns to 500 microns). As another example, in some embodiments in which the particles are used to embolize a uterine fibroid, the particles delivered to the subject can have an arithmetic mean maximum dimension of 1,200 microns or less (e.g., from 500 microns to 700 microns; from 700 microns to 900 microns; from 900 microns to 1,200 microns). As an additional example, in certain embodiments in which the particles are used to treat a neural condition (e.g., a brain tumor) and/or head trauma (e.g., bleeding in the head), the particles delivered to the subject can have an arithmetic mean maximum dimension of less than 100 microns (e.g., less than 50 microns). As a further example, in some embodiments in which the particles are used to treat a lung condition, the particles delivered to the subject can have an arithmetic mean maximum dimension of less than 100 microns (e.g., less than 50 microns). As another example, in certain embodiments in which the particles are used to treat thyroid cancer, the particles can have an arithmetic maximum dimension of 1,200 microns or less (e.g., from 1,000 microns to 1,200 microns). As an additional example, in some embodiments in which the particles are used only for therapeutic agent delivery, the particles can have an arithmetic mean maximum dimension of less than 100 microns (e.g., less than 50 microns, less than 10 microns, less than five microns).

The arithmetic mean maximum dimension of a group of particles can be determined using a Beckman Coulter RapidVUE Image Analyzer version 2.06 (Beckman Coulter, Miami, Fla.), described above. The arithmetic mean maximum dimension of a group of particles (e.g., in a composition) can be determined by dividing the sum of the diameters of all of the particles in the group by the number of particles in the group.

Additionally or alternatively to having pores, a particle can have one or more cavities. For example, a particle can be formed so that the polymer surrounds one or more cavities.

A pore has a maximum dimension of at least 0.0 1 micron (e.g., at least 0.05 micron, at least 0.1 micron, at least 0.5 micron, at least one micron, at least five microns, at least 10 microns, at least 15 microns, at least 20 microns, at least 25 microns, at least 30 microns, at least 35 microns, at least 50 microns, at least 100 microns, at least 150 microns, at least 200 microns, at least 250 microns), and/or at most 300 microns (e.g., at most 250 microns, at most 200 microns, at most 150 microns, at most 100 microns, at most 50 microns, at most 35 microns, at most 30 microns, at most 25 microns, at most 20 microns, at most 15 microns, at most 10 microns, at most five microns, at most one micron, at most 0.5 micron, at most 0.1 micron, at most 0.05 micron).

A cavity has a maximum dimension of at least one micron (e.g., a least five microns, at least 10 microns, at least 25 microns, at least 50 microns, at least 100 microns, at least 250 microns, at least 500 microns, at least 750 microns) and/or at most 1,000 microns (e.g., at most 750 microns, at most 500 microns, at most 250 microns, at most 100 microns, at most 50 microns, at most 25 microns, at most 10 microns, at most five microns). In some embodiments (e.g., when the particle is used to deliver a therapeutic agent within a body lumen, independent of whether embolization is desired), the particle can also include a therapeutic agent (e.g., in one or more pores, in one or more cavities, on the surface of the particle).

Therapeutic agents include genetic therapeutic agents, non-genetic therapeutic agents, and cells, and can be negatively charged, positively charged, amphoteric, or neutral. Therapeutic agents can be, for example, materials that are biologically active to treat physiological conditions; pharmaceutically active compounds; proteins; gene therapies; nucleic acids with and without carrier vectors (e.g., recombinant nucleic acids, DNA (e.g., naked DNA), cDNA, RNA, genomic DNA, cDNA or RNA in a non-infectious vector or in a viral vector which may have attached peptide targeting sequences, antisense nucleic acids (RNA, DNA)); oligonucleotides; gene/vector systems (e.g., anything that allows for the uptake and expression of nucleic acids); DNA chimeras (e.g., DNA chimeras which include gene sequences and encoding for ferry proteins such as membrane translocating sequences (“MTS”) and herpes simplex virus-1 (“VP22”)); compacting agents (e.g., DNA compacting agents); viruses; polymers; hyaluronic acid; proteins (e.g., enzymes such as ribozymes, asparaginase); immunologic species; nonsteroidal anti-inflammatory medications; oral contraceptives; progestins; gonadotrophin-releasing hormone agonists; chemotherapeutic agents; and radioactive species (e.g., radioisotopes, radioactive molecules). Examples of radioactive species include yttrium (⁹⁰Y), holmium (¹⁶⁶Ho), phosphorus (³²P), lutetium (¹⁷⁷Lu), actinium (²²⁵Ac), praseodymium, astatine (²¹¹At), rhenium (¹⁸⁶Re), bismuth (²¹²Bi or ²¹³Bi),), samarium (¹⁵³Sm), iridium (¹⁹²Ir), rhodium (105Rh), iodine (131I or ¹²⁵I), indium (¹¹¹In), technetium (⁹⁹Tc), phosphorus (³²P), sulfur (35S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), and/or gallium (67Ga). In some embodiments, yttrium (⁹⁰Y), lutetium (177Lu), actinium (²²⁵Ac), praseodymium, astatine (²¹¹At), rhenium (¹⁸⁶Re), bismuth (²¹²Bi or ²¹³Bi), holmium (¹⁶⁶Ho), samarium (¹⁵³Sm), iridium (¹⁹²Ir), and/or rhodium (¹⁰⁵Rh) can be used as therapeutic agents. In certain embodiments, yttrium (⁹⁰Y), lutetium (¹⁷⁷Lu), actinium (²²⁵ Ac), praseodymium, astatine (²¹¹At), rhenium (¹⁸⁶Re), bismuth (²¹²Bi or ²¹³Bi), holmium (¹⁶⁶Ho), samarium (¹⁵³Sm), iridium (¹⁹²Ir), rhodium (¹⁰⁵Rh), iodine (¹³¹I or ¹²⁵I), indium (¹¹¹In), technetium (⁹⁹Tc), phosphorus (³²P), carbon (¹⁴C), and/or tritium (³H) can be used as a radioactive label (e.g., for use in diagnostics). In some embodiments, a radioactive species can be a radioactive molecule that includes antibodies containing one or more radioisotopes, for example, a radiolabeled antibody. Radioisotopes that can be bound to antibodies include, for example, iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹Tc), phosphorus (³²P), rhodium (¹⁰⁵Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), and/or gallium (⁶⁷Ga). Examples of antibodies include monoclonal and polyclonal antibodies including RS7, Mov18, MN-14 IgG, CC49, COL-1, mAB A33, NP-4 F(ab′)2 anti-CEA, anti-PSMA, ChL6, m-170, or antibodies to CD20, CD74 or CD52 antigens. Examples of radioisotope/antibody pairs include m-170 MAB with ⁹⁰Y. Examples of commercially available radioisotope/antibody pairs include Zevalin™ (IDEC pharmaceuticals, San Diego, Calif.) and Bexxar™ (Corixa corporation, Seattle, Wash.). Further examples of radioisotope/antibody pairs can be found in J. Nucl. Med. 2003, Apr: 44(4): 632-40.

Non-limiting examples of therapeutic agents include anti-thrombogenic agents; thrombogenic agents; agents that promote clotting; agents that inhibit clotting; antioxidants; angiogenic and anti-angiogenic agents and factors; anti-proliferative agents (e.g., agents capable of blocking smooth muscle cell proliferation, such as rapamycin); calcium entry blockers (e.g., verapamil, diltiazem, nifedipine); targeting factors (e.g., polysaccharides, carbohydrates); agents that can stick to the vasculature (e.g., charged moieties) (e.g., gelatin, chitosn, collagen, polymers containing bioactive groups like RGD peptides); and survival genes which protect against cell death (e.g., anti-apoptotic Bcl-2 family factors and Akt kinase).

Examples of non-genetic therapeutic agents include: anti-thrombotic agents such as heparin, heparin derivatives, urokinase, and PPack (dextrophenylalanine proline arginine chloromethylketone); anti-inflammatory agents such as dexamethasone, prednisolone, corticosterone, budesonide, estrogen, acetyl salicylic acid, sulfasalazine and mesalamine; antineoplastic/antiproliferative/anti-mitotic agents such as paclitaxel, 5-fluorouracil, cisplatin, methotrexate, doxorubicin, vinblastine, vincristine, epothilones, endostatin, angiostatin, angiopeptin, monoclonal antibodies capable of blocking smooth muscle cell proliferation, and thymidine kinase inhibitors; anesthetic agents such as lidocaine, bupivacaine and ropivacaine; anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet factors or peptides; vascular cell growth promoters such as growth factors, transcriptional activators, and translational promoters; vascular cell growth inhibitors such as growth factor inhibitors (e.g., PDGF inhibitor-Trapidil), growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; protein kinase and tyrosine kinase inhibitors (e.g., tyrphostins, genistein, quinoxalines); prostacyclin analogs; cholesterol-lowering agents; angiopoietins; antimicrobial agents such as triclosan, cephalosporins, aminoglycosides and nitrofurantoin; cytotoxic agents, cytostatic agents and cell proliferation affectors; vasodilating agents; and agents that interfere with endogenous vasoactive mechanisms.

Examples of genetic therapeutic agents include: anti-sense DNA and RNA; DNA coding for anti-sense RNA, tRNA or rRNA to replace defective or deficient endogenous molecules, angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor a, hepatocyte growth factor, and insulin like growth factor, cell cycle inhibitors including CD inhibitors, thymidine kinase (“TK”) and other agents useful for interfering with cell proliferation, and the family of bone morphogenic proteins (“BMP's”), including BMP2, BMP3, BMP4, BMP5, BMP6 (Vgr1), BMP7 (OP1), BMP8, BMP9, BMP10, BM11, BMP12, BMP13, BMP14, BMP15, and BMP16. Currently preferred BMP's are any of BMP2, BMP3, BMP4, BMP5, BMP6 and BMP7. These dimeric proteins can be provided as homodimers, heterodimers, or combinations thereof, alone or together with other molecules. Alternatively or additionally, molecules capable of inducing an upstream or downstream effect of a BMP can be provided. Such molecules include any of the “hedgehog” proteins, or the DNA's encoding them. Vectors of interest for delivery of genetic therapeutic agents include: plasmids; viral vectors such as adenovirus (AV), adenoassociated virus (AAV) and lentivirus; and non-viral vectors such as lipids, liposomes and cationic lipids.

Cells include cells of human origin (autologous or allogeneic), including stem cells, or from an animal source (xenogeneic), which can be genetically engineered if desired to deliver proteins of interest.

Several of the above and numerous additional therapeutic agents are disclosed in Kunz et al., U.S. Pat. No. 5,733,925, which is incorporated herein by reference. Therapeutic agents disclosed in this patent include the following:

“Cytostatic agents” (i.e., agents that prevent or delay cell division in proliferating cells, for example, by inhibiting replication of DNA or by inhibiting spindle fiber formation). Representative examples of cytostatic agents include modified toxins, methotrexate, adriamycin, radionuclides (e.g., such as disclosed in Fritzberg et al., U.S. Pat. No. 4,897,255), protein kinase inhibitors, including staurosporin, a protein kinase C inhibitor of the following formula:

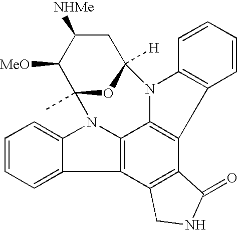

as well as diindoloalkaloids having one of the following general structures:

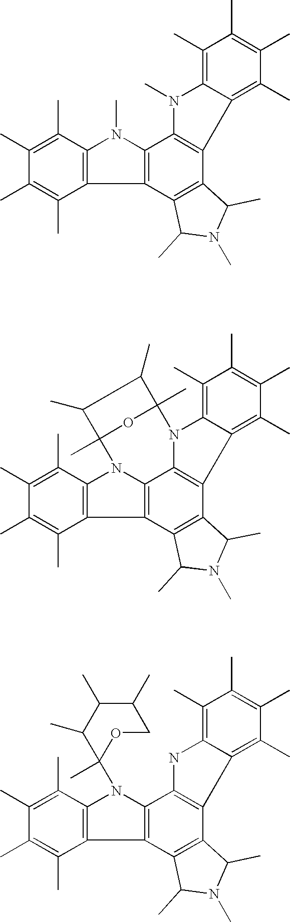

as well as stimulators of the production or activation of TGF-beta, including Tamoxifen and derivatives of functional equivalents (e.g., plasmin, heparin, compounds capable of reducing the level or inactivating the lipoprotein Lp(a) or the glycoprotein apolipoprotein(a)) thereof, TGF-beta or functional equivalents, derivatives or analogs thereof, suramin, nitric oxide releasing compounds (e.g., nitroglycerin) or analogs or functional equivalents thereof, paclitaxel or analogs thereof (e.g., taxotere), inhibitors of specific enzymes (such as the nuclear enzyme DNA topoisomerase II and DNA polymerase, RNA polymerase, adenyl guanyl cyclase), superoxide dismutase inhibitors, terminal deoxynucleotidyl-transferase, reverse transcriptase, antisense oligonucleotides that suppress smooth muscle cell proliferation and the like. Other examples of “cytostatic agents” include peptidic or mimetic inhibitors (i.e., antagonists, agonists, or competitive or non-competitive inhibitors) of cellular factors that may (e.g., in the presence of extracellular matrix) trigger proliferation of smooth muscle cells or pericytes: e.g., cytokines (e.g., interleukins such as IL-1), growth factors (e.g., PDGF, TGF-alpha or -beta, tumor necrosis factor, smooth muscle- and endothelial-derived growth factors, i.e., endothelin, FGF), homing receptors (e.g., for platelets or leukocytes), and extracellular matrix receptors (e.g., integrins). Representative examples of useful therapeutic agents in this category of cytostatic agents addressing smooth muscle proliferation include: subfragments of heparin, triazolopyrimidine (trapidil; a PDGF antagonist), lovastatin, and prostaglandins E1 or I2.

Agents that inhibit the intracellular increase in cell volume (i.e., the tissue volume occupied by a cell), such as cytoskeletal inhibitors or metabolic inhibitors. Representative examples of cytoskeletal inhibitors include colchicine, vinblastin, cytochalasins, paclitaxel and the like, which act on microtubule and microfilament networks within a cell. Representative examples of metabolic inhibitors include staurosporin, trichothecenes, and modified diphtheria and ricin toxins, Pseudomonas exotoxin and the like. Trichothecenes include simple trichothecenes (i.e., those that have only a central sesquiterpenoid structure) and macrocyclic trichothecenes (i.e., those that have an additional macrocyclic ring), e.g., a verrucarins or roridins, including Verrucarin A, Verrucarin B, Verrucarin J (Satratoxin C), Roridin A, Roridin C, Roridin D, Roridin E (Satratoxin D), Roridin H.

Agents acting as an inhibitor that blocks cellular protein synthesis and/or secretion or organization of extracellular matrix (i.e., an “anti-matrix agent”). Representative examples of “anti-matrix agents” include inhibitors (i.e., agonists and antagonists and competitive and non-competitive inhibitors) of matrix synthesis, secretion and assembly, organizational cross-linking (e.g., transglutaminases cross-linking collagen), and matrix remodeling (e.g., following wound healing). A representative example of a useful therapeutic agent in this category of anti-matrix agents is colchicine, an inhibitor of secretion of extracellular matrix. Another example is tamoxifen for which evidence exists regarding its capability to organize and/or stabilize as well as diminish smooth muscle cell proliferation following angioplasty. The organization or stabilization may stem from the blockage of vascular smooth muscle cell maturation in to a pathologically proliferating form.

Agents that are cytotoxic to cells, particularly cancer cells. Preferred agents are Roridin A, Pseudomonas exotoxin and the like or analogs or functional equivalents thereof. A plethora of such therapeutic agents, including radioisotopes and the like, have been identified and are known in the art. In addition, protocols for the identification of cytotoxic moieties are known and employed routinely in the art.

A number of the above therapeutic agents and several others have also been identified as candidates for vascular treatment regimens, for example, as agents targeting restenosis. Such agents include one or more of the following: calcium-channel blockers, including benzothiazapines (e.g., diltiazem, clentiazem); dihydropyridines (e.g., nifedipine, amlodipine, nicardapine); phenylalkylamines (e.g., verapamil); serotonin pathway modulators, including 5-HT antagonists (e.g., ketanserin, naftidrofuryl) and 5-HT uptake inhibitors (e.g., fluoxetine); cyclic nucleotide pathway agents, including phosphodiesterase inhibitors (e.g., cilostazole, dipyridamole), adenylate/guanylate cyclase stimulants (e.g., forskolin), and adenosine analogs; catecholamine modulators, including a-antagonists (e.g., prazosin, bunazosine), β-antagonists (e.g., propranolol), and α/β-antagonists (e.g., labetalol, carvedilol); endothelin receptor antagonists; nitric oxide donors/releasing molecules, including organic nitrates/nitrites (e.g., nitroglycerin, isosorbide dinitrate, amyl nitrite), inorganic nitroso compounds (e.g., sodium nitroprusside), sydnonimines (e.g., molsidomine, linsidomine), nonoates (e.g., diazenium diolates, NO adducts of alkanediamines), S-nitroso compounds, including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), C-nitroso-, O-nitroso- and N-nitroso-compounds, and L-arginine; ACE inhibitors (e.g., cilazapril, fosinopril, enalapril); ATII-receptor antagonists (e.g., saralasin, losartin); platelet adhesion inhibitors (e.g., albumin, polyethylene oxide); platelet aggregation inhibitors, including aspirin and thienopyridine (ticlopidine, clopidogrel) and GP Iib/IIIa inhibitors (e.g., abciximab, epitifibatide, tirofiban, intergrilin); coagulation pathway modulators, including heparinoids (e.g., heparin, low molecular weight heparin, dextran sulfate, β-cyclodextrin tetradecasulfate), thrombin inhibitors (e.g., hirudin, hirulog, PPACK (D-phe-L-propyl-L-arg-chloromethylketone), argatroban), Fxa inhibitors (e.g., antistatin, TAP (tick anticoagulant peptide)), vitamin K inhibitors (e.g., warfarin), and activated protein C; cyclooxygenase pathway inhibitors (e.g., aspirin, ibuprofen, flurbiprofen, indomethacin, sulfinpyrazone); natural and synthetic corticosteroids (e.g., dexamethasone, prednisolone, methprednisolone, hydrocortisone); lipoxygenase pathway inhibitors (e.g., nordihydroguairetic acid, caffeic acid; leukotriene receptor antagonists; antagonists of E- and P-selectins; inhibitors of VCAM-1 and ICAM-1 interactions; prostaglandins and analogs thereof, including prostaglandins such as PGE1 and PGI2; prostacyclins and prostacyclin analogs (e.g., ciprostene, epoprostenol, carbacyclin, iloprost, beraprost); macrophage activation preventers (e.g., bisphosphonates); HMG-CoA reductase inhibitors (e.g., lovastatin, pravastatin, fluvastatin, simvastatin, cerivastatin); fish oils and omega-3-fatty acids; free-radical scavengers/antioxidants (e.g., probucol, vitamins C and E, ebselen, retinoic acid (e.g., trans-retinoic acid), SOD mimics); agents affecting various growth factors including FGF pathway agents (e.g., bFGF antibodies, chimeric fusion proteins), PDGF receptor antagonists (e.g., trapidil), IGF pathway agents (e.g., somatostatin analogs such as angiopeptin and ocreotide), TGF-β pathway agents such as polyanionic agents (heparin, fucoidin), decorin, and TGF-β antibodies, EGF pathway agents (e.g., EGF antibodies, receptor antagonists, chimeric fusion proteins), TNF-α pathway agents (e.g., thalidomide and analogs thereof), thromboxane A2 (TXA2) pathway modulators (e.g., sulotroban, vapiprost, dazoxiben, ridogrel), protein tyrosine kinase inhibitors (e.g., tyrphostin, genistein, and quinoxaline derivatives); MMP pathway inhibitors (e.g., marimastat, ilomastat, metastat), and cell motility inhibitors (e.g., cytochalasin B); antiproliferative/antineoplastic agents including antimetabolites such as purine analogs (e.g., 6-mercaptopurine), pyrimidine analogs (e.g., cytarabine and 5-fluorouracil) and methotrexate, nitrogen mustards, alkyl sulfonates, ethylenimines, antibiotics (e.g., daunorubicin, doxorubicin, daunomycin, bleomycin, mitomycin, penicillins, cephalosporins, ciprofalxin, vancomycins, aminoglycosides, quinolones, polymyxins, erythromycins, tertacyclines, chloramphenicols, clindamycins, linomycins, sulfonamides, and their homologs, analogs, fragments, derivatives, and pharmaceutical salts), nitrosoureas (e.g., carmustine, lomustine) and cisplatin, agents affecting microtubule dynamics (e.g., vinblastine, vincristine, colchicine, paclitaxel, epothilone), caspase activators, proteasome inhibitors, angiogenesis inhibitors (e.g., endostatin, angiostatin and squalamine), and rapamycin, cerivastatin, flavopiridol and suramin; matrix deposition/organization pathway inhibitors (e.g., halofuginone or other quinazolinone derivatives, tranilast); endothelialization facilitators (e.g., VEGF and RGD peptide); and blood rheology modulators (e.g., pentoxifylline).

Other examples of therapeutic agents include anti-tumor agents, such as docetaxel, alkylating agents (e.g., mechlorethamine, chlorambucil, cyclophosphamide, melphalan, ifosfamide), plant alkaloids (e.g., etoposide), inorganic ions (e.g., cisplatin), biological response modifiers (e.g., interferon), and hormones (e.g., tamoxifen, flutamide), as well as their homologs, analogs, fragments, derivatives, and pharmaceutical salts.

Additional examples of therapeutic agents include organic-soluble therapeutic agents, such as mithramycin, cyclosporine, and plicamycin. Further examples of therapeutic agents include pharmaceutically active compounds, anti-sense genes, viral, liposomes and cationic polymers (e.g., selected based on the application), biologically active solutes (e.g., heparin), prostaglandins, prostcyclins, L-arginine, nitric oxide (NO) donors (e.g., lisidomine, molsidomine, NO-protein adducts, NO-polysaccharide adducts, polymeric or oligomeric NO adducts or chemical complexes), enoxaparin, Warafin sodium, dicumarol, interferons, interleukins, chymase inhibitors (e.g., Tranilast), ACE inhibitors (e.g., Enalapril), serotonin antagonists, 5-HT uptake inhibitors, and beta blockers, and other antitumor and/or chemotherapy drugs, such as BiCNU, busulfan, carboplatinum, cisplatinum, cytoxan, DTIC, fludarabine, mitoxantrone, velban, VP-16, herceptin, leustatin, navelbine, rituxan, and taxotere.

In some embodiments, a therapeutic agent can be hydrophilic. An example of a hydrophilic therapeutic agent is doxorubicin hydrochloride. In certain embodiments, a therapeutic agent can be hydrophobic. Examples of hydrophobic therapeutic agents include paclitaxel, cisplatin, tamoxifen, and doxorubicin base. In some embodiments, a therapeutic agent can be lipophilic. Examples of lipophilic therapeutic agents include paclitaxel, other taxane derivative, dexamethasone, other steroid based therapeutics.

Therapeutic agents are described, for example, in DiMatteo et al., U.S. Patent Application Publication No. US 2004/0076582 A1, published on Apr. 22, 2004, and entitled “Agent Delivery Particle“; Schwarz et al., U.S. Pat. No. 6,368,658; Buiser et al., U.S. patent application Ser. No. 11/311,617, filed on Dec. 19, 2005, and entitled “Coils“; and Song, U.S. patent application Ser. No. 11/355,301, filed on Feb. 15, 2006, and entitled “Block Copolymer Particles”, all of which are incorporated herein by reference. In certain embodiments, in addition to or as an alternative to including therapeutic agents, particle 100 can include one or more radiopaque materials, materials that are visible by magnetic resonance imaging (MRI-visible materials), ferromagnetic materials, and/or contrast agents (e.g., ultrasound contrast agents). These materials can, for example, be bonded to the chemical species (monomer(s), oligomers(s), polymer(s)). Radiopaque materials, MRI-visible materials, ferromagnetic materials, and contrast agents are described, for example, in Rioux et al., U.S. Patent Application Publication No. US 2004/0101564 A1, published on May 27, 2004, and entitled “Embolization”, which is incorporated herein by reference.

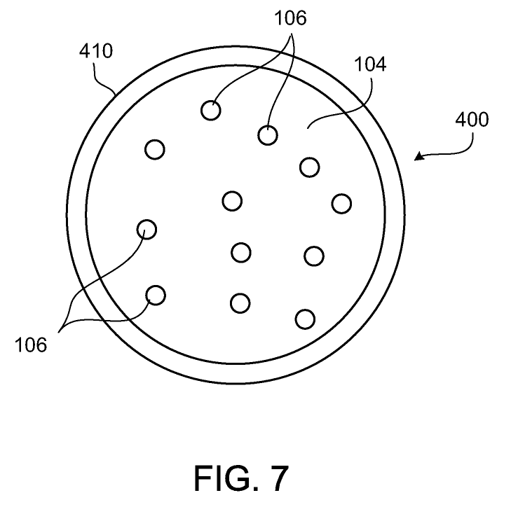

In certain embodiments, a particle can also include a coating. For example, FIG. 7 shows a particle 400 having a matrix 104, pores 106 and, and a coating 410. Coating 410 can, for example, be formed of a polymer (e.g., alginate) that is different from the polymer in matrix 104. Coating 410 can, for example, regulate release of therapeutic agent from particle 400, and/or provide protection to the interior region of particle 400 (e.g., during delivery of particle 400 to a target site). In certain embodiments, coating 410 can be formed of a bioerodible and/or bioabsorbable material that can erode and/or be absorbed as particle 400 is delivered to a target site. This can, for example, allow the interior region of particle 400 to deliver a therapeutic agent to the target site once particle 400 has reached the target site. A bioerodible material can be, for example, a polysaccharide (e.g., alginate); a polysaccharide derivative; an inorganic, ionic salt; a water soluble polymer (e.g., polyvinyl alcohol, such as polyvinyl alcohol that has not been cross-linked); biodegradable poly DL-lactide-poly ethylene glycol (PELA); a hydrogel (e.g., polyacrylic acid, hyaluronic acid, gelatin, carboxymethyl cellulose); a polyethylene glycol (PEG); chitosan; a polyester (e.g., a polycaprolactone); a poly(ortho ester); a polyanhydride; a poly(lactic-co-glycolic) acid (e.g., a poly(d-lactic-co-glycolic) acid); a poly(lactic acid) (PLA); a poly(glycolic acid) (PGA); or a combination thereof. In some embodiments, coating 410 can be formed of a swellable material, such as a hydrogel (e.g., polyacrylamide co-acrylic acid). The swellable material can be made to swell by, for example, changes in pH, temperature, and/or salt. In certain embodiments in which particle 400 is used in an embolization procedure, coating 410 can swell at a target site, thereby enhancing occlusion of the target site by particle 400.

In some embodiments, the coating can be porous. The coating can, for example, be formed of one or more of the above-disclosed polymers.

In certain embodiments, a particle can include a coating that includes one or more therapeutic agents (e.g., a relatively high concentration of one or more therapeutic agents). One or more of the therapeutic agents can also be loaded into the interior region of the particle. Thus, the surface of the particle can release an initial dosage of therapeutic agent, after which the interior region of the particle can provide a burst release of therapeutic agent. The therapeutic agent on the surface of the particle can be the same as or different from the therapeutic agent in the interior region of the particle. The therapeutic agent on the surface of the particle can be applied to the particle by, for example, exposing the particle to a high concentration solution of the therapeutic agent.

In some embodiments, a therapeutic agent coated particle can include another coating over the surface of the therapeutic agent (e.g., a bioerodible polymer which erodes when the particle is administered). The coating can assist in controlling the rate at which therapeutic agent is released from the particle. For example, the coating can be in the form of a porous membrane. The coating can delay an initial burst of therapeutic agent release. In certain embodiments, the coating can be applied by dipping and/or spraying the particle. The bioerodible polymer can be a polysaccharide (e.g., alginate). In some embodiments, the coating can be an inorganic, ionic salt. Other examples of bioerodible coating materials include polysaccharide derivatives, water-soluble polymers (such as polyvinyl alcohol, e.g., that has not been cross-linked), biodegradable poly DL-lactide-poly ethylene glycol (PELA), hydrogels (e.g., polyacrylic acid, hyaluronic acid, gelatin, carboxymethyl cellulose), polyethylene glycols (PEG), chitosan, polyesters (e.g., polycaprolactones), poly(ortho esters), polyanhydrides, poly(lactic acids) (PLA), polyglycolic acids (PGA), poly(lactic-co-glycolic) acids (e.g., poly(d-lactic-co-glycolic) acids), and combinations thereof. The coating can include therapeutic agent or can be substantially free of therapeutic agent. The therapeutic agent in the coating can be the same as or different from an agent on a surface layer of the particle and/or within the particle. A polymer coating (e.g., a bioerodible coating) can be applied to the particle surface in embodiments in which a high concentration of therapeutic agent has not been applied to the particle surface. Coatings are described, for example, in DiMatteo et al., U.S. Patent Application Publication No. US 2004/0076582 A1, published on Apr. 22, 2004, and entitled “Agent Delivery Particle”, which is incorporated herein by reference.

The following examples are illustrative only and not intended as limiting.

### EXAMPLES

### Freeze-Drying

PVA-containing particles were prepared using freeze-drying techniques as follows. A PVA-alginate solution was prepared by mixing 8-10.5 grams of PVA, 0.75-2 grams of alginate, and 100 grams of water. A syringe containing the PVA-alginate solution was immersed in a 65° C. water bath for 1 hour. A 900-1200 nm tip was put on a droplet generator (NISCO Encapsulation unit VAR D), and the drop generator was flushed with 120 milliliters of 65° C. deionized water. A 26.6 centimeter diameter syringe pump with pressure set to 15 milliliter/minute was then used to input the PVA-alginate solution to the droplet generator with the waveform generator set to 300-700 kHz (depending on the desired particle size) and an amplitude of 10V. The droplet generator discharged a stream of droplets of the PVA-alginate solution into a 500-milliliter beaker containing 150 milliliters of calcium chloride solution (2-4% weight percent calcium chloride in water). During collection, the beaker was placed on a stir-plate spinning a magnetic stir-bar in the beaker at approximately 100 rpm. A strainer was used to separate the resulting microspheres from the calcium chloride solution.

The resulting particles were subjected to one or more freeze-drying cycles. In each freeze-drying cycle, where each cycle was composed of: freezing (directly or indirectly) the particles to by immersion in liquid nitrogen, placing the particles in the freeze-dryer for 24 hours, and thawing the particles at room temperature for 24 hours.

A portion of the resulting particles were then submerged in a sodium hexametaphosphate solution (5% w/v in water) to remove the alginate. The sodium hexametaphosphate solution with the particles was stirred at 200 rpm for 30 minutes. The sodium hexametaphosphate solution was then strained to remove the particles. The particles were then rinsed in deionized water using two rinse cycles of shaking with deionized water at 200 rpm for 30 minutes. The amounts of PVA retained and of sodium alginate removed were measured and recorded. The theoretical amount of PVA initially present after freeze-drying was calculated by subtracting the amount of sodium alginate removed from the initial weight of the freeze-dried particles. The percent retention of PVA in the particles was calculated by dividing the measured amount of PVA retained by the calculated amount of PVA initially present after freeze-drying.

Table 1 summarizes the results of these experiments. It was observed that the number of freeze-drying cycles applied to the particles impacted the amount of PVA that was removed with the alginate. The percent retention of PVA retained significantly increased with the application of 4 or more cycles of freeze-drying.

TABLE 1
Summary of Experimental Results (Freeze-Drying)

| | Initial | | | | Retention |
|---|---|---|---|---|---|
| # of | weight | PVA retained | Na Alginate | Calculated | of |
| cycles | (mg) | (mg) | (mg) | PVA (mg) | PVA (%) |
| 1 | 1051.30 | 210.6 | 263.23 | 788.07 | 26.72 |
| 2 | 300.05 | 42.85 | 75.13 | 224.92 | 19.05 |
| 3 | 306.60 | 48.35 | 76.77 | 229.83 | 21.04 |
| 4 | 301.15 | 145.10 | 75.40 | 225.75 | 64.28 |
| 5 | 310.10 | 144.25 | 77.65 | 232.45 | 62.05 |

Freeze-drying changed the shape of the particles from generally spherical to irregular shapes. Rehydration of the particles by immersion in water to some extent restored smooth contours to outer surfaces of the particles but generally did not restore the particles to their pre-freeze-drying spherical shapes. As part of the freeze-drying process, some microspheres were directly frozen (i.e., by immersion in liquid nitrogen) and some microspheres were indirectly frozen (i.e., placed in glass vials which were then immersed in liquid nitrogen). The indirectly frozen particles generally remained more spherical than the directly frozen particles.

### Radiation

PVA-containing particles were also prepared using techniques including irradiation of the particles as follows. PVA-alginate microspheres were produced using generally the same procedure as described above. A single freeze-drying cycle was then applied to the microspheres. After freeze-drying, nitrogen gas was used to purge oxygen from the container holding the microspheres and samples of the microspheres were irradiated with electron beam radiation at dosages of 25 kilograys, 50 kilograys, and 100 kilograys. Alginate was removed from the irradiated microspheres using substantially the same procedure as described above. The particles were then frozen and a freeze-drying cycle applied before the final weight of the particles was measured. It was not clear whether irradiating the samples increased PVA retention.

### Other Embodiments

While certain embodiments have been described, other embodiments are possible.

As one example, in some embodiments, a polymer (e.g., PVA) solution is placed into a mold and formed into a film. The film is frozen and then irradiated with, for example, electron beam or gamma radiation. The film is then be shredded to form particles. Other forms of radiation can also be used.

As another example, in some embodiments, particles can be used for tissue bulking. As an example, the particles can be placed (e.g., injected) into tissue adjacent to a body passageway. The particles can narrow the passageway, thereby providing bulk and allowing the tissue to constrict the passageway more easily. The particles can be placed in the tissue according to a number of different methods, for example, percutaneously, laparoscopically, and/or through a catheter. In certain embodiments, a cavity can be formed in the tissue, and the particles can be placed in the cavity. Particle tissue bulking can be used to treat, for example, intrinsic sphincteric deficiency (ISD), vesicoureteral reflux, gastroesophageal reflux disease (GERD), and/or vocal cord paralysis (e.g., to restore glottic competence in cases of paralytic dysphonia). In some embodiments, particle tissue bulking can be used to treat urinary incontinence and/or fecal incontinence. The particles can be used as a graft material or a filler to fill and/or to smooth out soft tissue defects, such as for reconstructive or cosmetic applications (e.g., surgery). Examples of soft tissue defect applications include cleft lips, scars (e.g., depressed scars from chicken pox or acne scars), indentations resulting from liposuction, wrinkles (e.g., glabella frown wrinkles), and soft tissue augmentation of thin lips. Tissue bulking is described, for example, in Bourne et al., U.S. Patent Application Publication No. US 2003/0233150 A1, published on Dec. 18, 2003, and entitled “Tissue Treatment”, which is incorporated herein by reference.

As an additional example, in certain embodiments, particles can be used to treat trauma and/or to fill wounds. In some embodiments, the particles can include one or more bactericidal agents and/or bacteriostatic agents.

As a further example, while compositions including particles suspended in at least one carrier fluid have been described, in certain embodiments, particles may not be suspended in any carrier fluid. For example, particles alone can be contained within a syringe, and can be injected from the syringe into tissue during a tissue ablation procedure and/or a tissue bulking procedure.

As an additional example, in some embodiments, particles having different shapes, sizes, physical properties, and/or chemical properties can be used together in a procedure (e.g., an embolization procedure). The different particles can be delivered into the body of a subject in a predetermined sequence or simultaneously. In certain embodiments, mixtures of different particles can be delivered using a multi-lumen catheter and/or syringe. In some embodiments, particles having different shapes and/or sizes can be capable of interacting synergistically (e.g., by engaging or interlocking) to form a well-packed occlusion, thereby enhancing embolization. Particles with different shapes, sizes, physical properties, and/or chemical properties, and methods of embolization using such particles are described, for example, in Bell et al., U.S. Patent Application Publication No. US 2004/0091543 A1, published on May 13, 2004, and entitled “Embolic Compositions”, and in DiCarlo et al., U.S. Patent Application Publication No. US 2005/0095428 A1, published on May 5, 2005, and entitled “Embolic Compositions”, both of which are incorporated herein by reference.

As a further example, in some embodiments in which a particle including a polymer is used for embolization, the particle can also include (e.g., encapsulate) one or more embolic agents, such as a sclerosing agent (e.g., ethanol), a liquid embolic agent (e.g., n-butyl-cyanoacrylate), and/or a fibrin agent. The other embolic agent(s) can enhance the restriction of blood flow at a target site.

As another example, in some embodiments, a treatment site can be occluded by using particles in conjunction with other occlusive devices. For example, particles can be used in conjunction with coils. Coils are described, for example, in Elliott et al., U.S. patent application Ser. No. 11/000,741, filed on Dec. 1, 2004, and entitled “Embolic Coils”, and in Buiser et al., U.S. patent application Ser. No. 11/311,617, filed on Dec. 19, 2005, and entitled “Coils”, both of which are incorporated herein by reference. In certain embodiments, particles can be used in conjunction with one or more gels. Gels are described, for example, in Richard et al., U.S. Patent Application Publication No. US 2006/0045900 A1, published on Mar. 2, 2006, and entitled “Embolization”, which is incorporated herein by reference. Additional examples of materials that can be used in conjunction with particles to treat a target site in a body of a subject include gel foams, glues, oils, and alcohol.

As a further example, while particles including a polymer have been described, in some embodiments, other types of medical devices and/or therapeutic agent delivery devices can include such a polymer. For example, in some embodiments, a coil can include a polymer as described above. In certain embodiments, the coil can be formed by flowing a stream of the polymer into an aqueous solution, and stopping the flow of the polymer stream once a coil of the desired length has been formed. Coils are described, for example, in Elliott et al., U.S. patent application Ser. No. 11/000,741, filed on Dec. 1, 2004, and entitled “Embolic Coils”, and in Buiser et al., U.S. patent application Ser. No. 11/311,617, filed on Dec. 19, 2005, and entitled “Coils”, both of which are incorporated herein by reference. In certain embodiments, sponges (e.g., for use as a hemostatic agent and/or in reducing trauma) can include a polymer as described above. In some embodiments, coils and/or sponges can be used as bulking agents and/or tissue support agents in reconstructive surgeries (e.g., to treat trauma and/or congenital defects).

Other embodiments are in the claims.

What is claimed is:

1. A method comprising:
freeze-drying a mixture comprising a polymer to form a particle.

2. The method of claim 1, wherein the polymer comprises polyvinyl alcohol.

3. The method of claim 1, wherein freeze-drying the mixture comprises reducing a pressure applied to the mixture to less than 500 millitorrs.

4. The method of claim 3, wherein reducing the pressure applied to the mixture comprises reducing the pressure applied to the mixture to less than 500 millitorrs and more than 10 millitorrs.

5. The method of claim 1, wherein freeze-drying the mixture comprises reducing the temperature applied to the mixture to less than −20° C.

6. The method of claim 5, wherein freeze-drying the mixture comprises reducing the temperature applied to the mixture to less than −50 ° C. and more than −100° C.

7. The method of claim 1, further comprising freeze-drying the mixture at least two times.

8. The method of claim 1, further comprising freeze-drying the mixture at least three times.

9. The method of claim 1, further comprising freeze-drying the mixture at least four times.

10. The method of claim 1, further comprising freeze-drying the mixture at least five times.

11. The method of claim 1, wherein freeze-drying the mixture comprises iteratively freeze-drying and thawing the mixture until the moisture content of the mixture is less than 5 percent.

12. The method of claim 11, wherein freeze-drying the mixture comprises iteratively freeze-drying and thawing the mixture until the moisture content of the mixture is less than 1 percent.

13. The method of claim 1, further comprising adding a therapeutic agent to the particle.

14. The method of claim 13, wherein adding the therapeutic agent to the particle comprises disposing the therapeutic agent into pores of the particle after the particle is formed.

15. The method of claim 13, wherein adding the therapeutic agent to the particle comprises adding the therapeutic agent to the mixture before freezing the mixture.

16. The method of claim 13, wherein adding the therapeutic agent to the particle comprises coating the particle with the therapeutic agent after the particle is formed.

17. The method of claim 1, further comprising adding a second polymer to the mixture.

18. The method of claim 1, further comprising irradiating the mixture.

19. A method comprising:
forming a particle having a maximum dimension of 5,000 microns by irradiating a polymer to crosslink the polymer.

20. The method of claim 19, further comprising freeze-drying the polymer.

21. The method of claim 19, wherein irradiating the polymer comprises irradiating the polymer with e-beam radiation.

22. The method of claim 19, wherein irradiating the polymer comprises irradiating the polymer with gamma radiation.

23. The method of claim 19, wherein irradiating the polymer comprises applying a dose of radiation from 20 to 110 kilograys to the polymer.

24. The method of claim 23, wherein irradiating the polymer comprises applying a dose of radiation from 25 to 100 kilograys to the polymer.

25. The method of claim 19, further comprising forming microspheres comprising the polymer before irradiating the polymer.

26. The method of claim 19, further comprising adding a therapeutic agent to the particle.

27. The method of claim 26, wherein adding the therapeutic agent to the particle comprises disposing the therapeutic agent into pores of the particle after the particle is formed.

28. The method of claim 26, wherein adding the therapeutic agent to the particle comprises mixing the therapeutic agent with the polymer before irradiating the polymer.

29. The method of claim 26, wherein adding the therapeutic agent to the particle comprises coating the particle with the therapeutic agent after the particle is formed.

30. The method of claim 19, further comprising placing the polymer in a mold to form a polymer sheet before irradiating the polymer.

31. The method of claim 30, further comprising grinding the polymer sheet to form particles after irradiating the polymer sheet.

32. The method of claim 19, further comprising placing the polymer in a container and purging oxygen from the container before irradiating the polymer.
